(19) United States
(12) Patent Application Publication
(10) Pub. No.: US 2021/0177367 A1
(43) Pub. Date: Jun. 17, 2021

(54) SYSTEMS AND METHODS FOR IMAGING THE THYROID

(71) Applicant: SHENZHEN XPECTVISION TECHNOLOGY CO., LTD., Shenzhen (CN)

(72) Inventors: Peiyan CAO, Shenzhen (CN); Yurun LIU, Shenzhen (CN)

(21) Appl. No.: 17/178,818

(22) Filed: Feb. 18, 2021

Related U.S. Application Data
(63) Continuation of application No. PCT/CN2018/104595, filed on Sep. 7, 2018.

(51) Int. Cl. A61B 6/00; H04N 5/32; A61B 6/06

(52) U.S. Cl. CPC A61B 6/485; H04N 5/32; A61B 6/481; A61B 6/50; A61B 6/4258; A61B 6/4233; A61B 6/4266; A61B 6/06; A61B 6/4241

(57) ABSTRACT

Disclosed herein is a system comprising: a plurality of X-ray detectors; wherein the X-ray detectors are configured to be positioned at different locations relative to the thyroid of a person, and to capture images of the thyroid with characteristic X-rays of iodine. Each of the X-ray detectors may comprise an array of pixels. The system may further comprise a collimator configured to limit fields of view of the pixels. Disclosed herein is a method comprising: causing emission of characteristic X-rays of iodine inside the thyroid of a person; capturing images of the thyroid with the characteristic X-rays, using a plurality of X-ray detectors positioned at different locations relative to the thyroid; determining a three-dimensional distribution of the iodine in the thyroid based on the images.

### BACKGROUND

X-ray fluorescence (XRF) is the emission of characteristic X-rays from a material that has been excited by, for example, exposure to high-energy X-rays or gamma rays. An electron on an inner orbital of an atom may be ejected, leaving a vacancy on the inner orbital, if the atom is exposed to X-rays or gamma rays with photon energy greater than the ionization potential of the electron. When an electron on an outer orbital of the atom relaxes to fill the vacancy on the inner orbital, an X-ray (fluorescent X-ray or secondary X-ray) is emitted. The emitted X-ray has a photon energy equal the energy difference between the outer orbital and inner orbital electrons.

For a given atom, the number of possible relaxations is limited. As shown in FIG. 1A, when an electron on the L orbital relaxes to fill a vacancy on the K orbital (L→K), the fluorescent X-ray is called Kα. The fluorescent X-ray from M→K relaxation is called Kβ. As shown in FIG. 1B, the fluorescent X-ray from M→L relaxation is called Lα, and so on.

### SUMMARY

Disclosed herein is a system comprising: a plurality of X-ray detectors; wherein the X-ray detectors are configured to be positioned at different locations relative to the thyroid of a person, and to capture images of the thyroid with characteristic X-rays of iodine.

According to an embodiment, the system further comprising a radiation source configured to irradiate the thyroid with radiation that causes iodine inside the thyroid to emit the characteristic X-rays.

According to an embodiment, each of the X-ray detectors comprises an array of pixels, and is configured to count numbers of photons of the characteristic X-rays incident on the pixels within a period of time.

According to an embodiment, each of the X-ray detectors may be configured to count the numbers of X-ray photons within a same period of time.

According to an embodiment, the pixels are configured to operate in parallel.

According to an embodiment, each of the pixels is configured to measure its dark current.

According to an embodiment, at least one of the X-ray detectors further comprises a collimator configured to limit fields of view of the pixels.

According to an embodiment, energies of particles of the radiation are in the range of 30-40 keV.

According to an embodiment, the radiation is X-ray or gamma ray.

According to an embodiment, at least one of the X-ray detectors comprises an X-ray absorption layer configured to generate an electrical signal responsive to photons of the characteristic X-rays incident thereon.

According to an embodiment, the X-ray absorption layer comprises silicon, germanium, GaAs, CdTe, CdZnTe, or a combination thereof.

According to an embodiment, the X-ray detectors do not comprise a scintillator.

According to an embodiment, the system further comprising a processor configured to determine a three-dimensional distribution of the iodine in the thyroid, based on the images

According to an embodiment, the iodine is not radioactive.

Disclosed herein is a method comprising: causing emission of characteristic X-rays of iodine inside the thyroid of a person; capturing images of the thyroid with the characteristic X-rays, using a plurality of X-ray detectors positioned at different locations relative to the thyroid; determining a three-dimensional distribution of the iodine in the thyroid based on the images.

According to an embodiment, causing emission of the characteristic X-rays comprises irradiating the thyroid with radiation that causes the emission of the characteristic X-rays.

According to an embodiment, the method further comprising introducing the iodine into the blood stream of the person.

According to an embodiment, capturing the images comprises counting numbers of photons of the characteristic X-rays within a period of time.

According to an embodiment, capturing the images comprises counting numbers of photons of the characteristic X-rays within a same period of time.

### BRIEF DESCRIPTION OF FIGURES

FIG. 1A and FIG. 1B schematically show mechanisms of XRF.

FIG. 2 schematically shows a system, according to an embodiment.

FIG. 3 schematically shows a side view of the system of FIG. 2, according to an embodiment.

FIG. 4 schematically shows an X-ray detector of the system of FIG. 2, according to an embodiment.

FIG. 5 schematically shows a cross-sectional view of the X-ray detector, according to an embodiment.

FIG. 6 schematically shows that the system of FIG. 2 may include a collimator 108, according to an embodiment.

FIG. 7 shows a flowchart for a method, according to an embodiment.

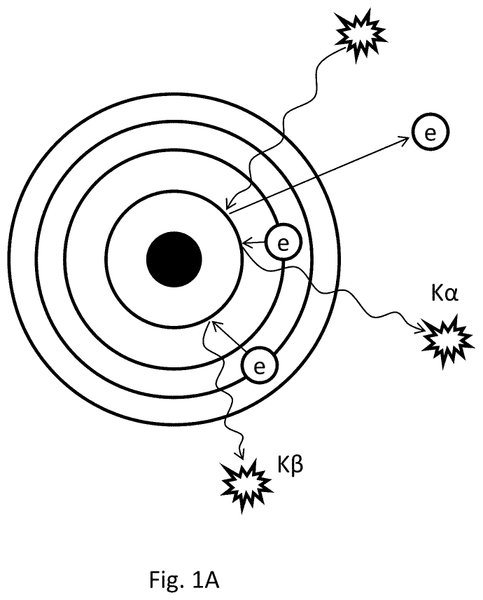

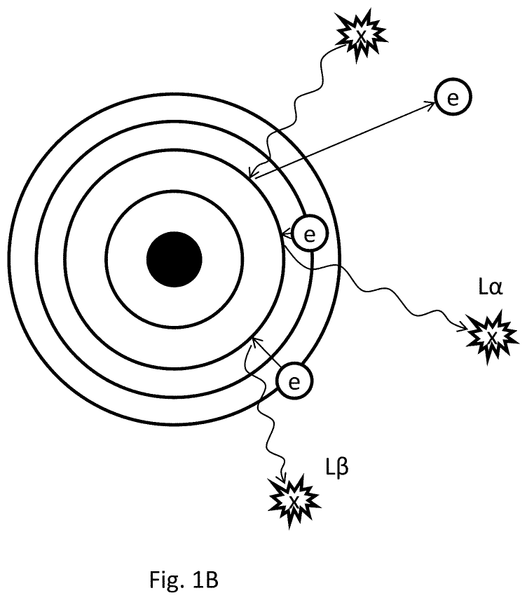

### DETAILED DESCRIPTION

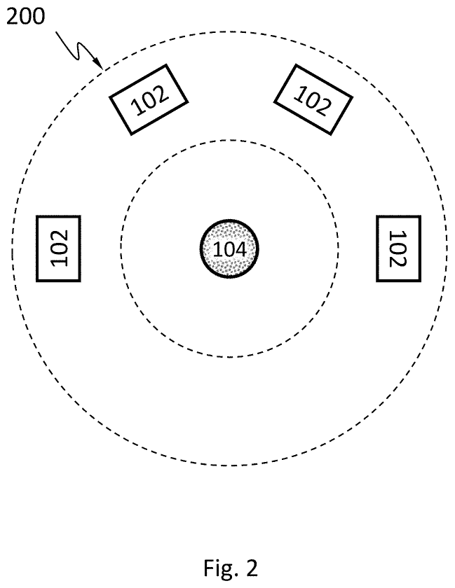

FIG. 2 schematically shows a system 200. The system 200 includes multiple X-ray detectors 102, according to an embodiment. The X-ray detectors 102 are positioned at different locations relative to an object 104 (e.g., the thyroid of a person). For example, the X-ray detectors 102 may be arranged at different locations along a semicircle around the person's neck or along the length of the person's neck. The X-ray detectors 102 may be arranged at about the same distance or different distances from the object 104. Other suitable arrangement of the X-ray detectors 102 may be possible. The X-ray detectors may be spaced equally or unequally apart in the angular direction. The positions of the X-ray detectors 102 are not necessarily fixed. For example, each of the X-ray detectors 102 may be movable towards and away from the object 104 or may be rotatable relative to the object 104.

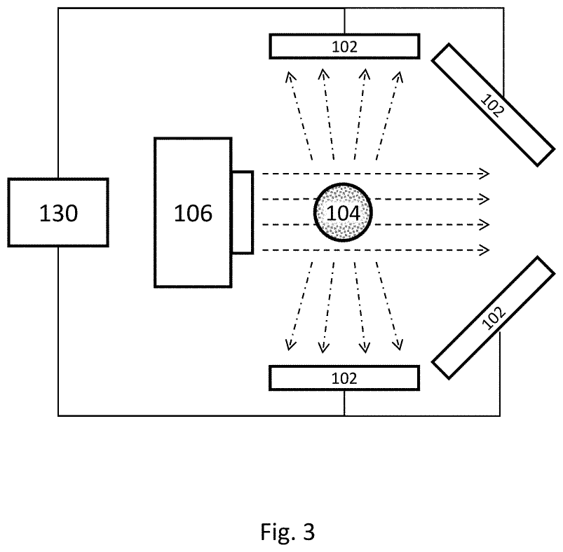

FIG. 3 schematically shows that the system 200 may include a radiation source 106, according to an embodiment. The system 200 may include more than one radiation source. The radiation source 106 irradiates the object 104 with radiation that can cause a chemical element (e.g., iodine) to emit characteristic X-rays (e.g., by fluorescence). The chemical element may not be radioactive. The radiation from the radiation source 106 may be X-ray or gamma ray. The energies of the particles of the radiation may be in the range of 30-40 keV. The radiation source 106 may be movable or stationary relative to the object 104. The X-ray detectors 102 form images of the object 104 with the characteristic X-rays, (e.g., by detecting the intensity distribution of the characteristic X-ray). The X-ray detectors 102 may be disposed at different locations around the object 104 where the X-ray detectors 102 do not receive the radiation from the radiation source 106 that is not scattered by the object 104. As shown in FIG. 3, the X-ray detectors 102 may avoid those positions where they would receive radiation from the radiation source 106 that has passed through the object 104. The X-ray detectors 102 may be movable or stationary relative to the object 104.

The object 104 may be a person or a portion (e.g., the thyroid) of a person. In an example, non-radioactive iodine is introduced into the person. The person may be directed to orally take or be injected a substance containing non-radioactive iodine. The non-radioactive iodine is absorbed by the thyroid. When the radiation from the radiation source 106 is directed toward the thyroid, the non-radioactive iodine inside the thyroid is excited by the radiation and emit the characteristic X-rays of iodine. The characteristic X-rays of iodine may include the K lines, or the K lines and the L lines. The X-ray detectors 102 capture images of the thyroid with the characteristic X-rays of iodine. The X-ray detectors 102 may disregard X-rays with energies different from characteristic X-rays of iodine. Spatial (e.g., three-dimensional) distribution of the iodine in the thyroid may be determined from these images. For example, the system 200 may have a processor 130 configured to determine the three-dimensional distribution of iodine in the thyroid, based on these images.

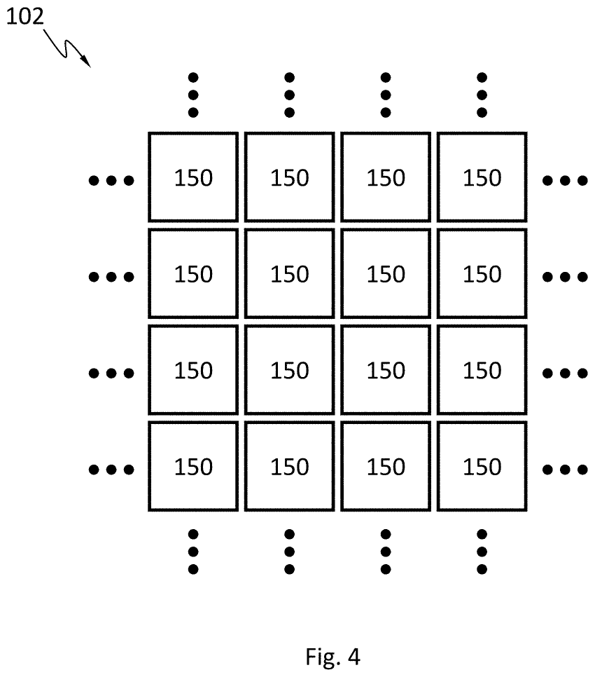

FIG. 4 schematically shows one of the X-ray detectors 102, according to an embodiment. The X-ray detector 102 has an array of pixels 150. The array may be a rectangular array, a honeycomb array, a hexagonal array or any other suitable array. Each pixel 150 is configured to count numbers of photons of X-rays (e.g., the characteristic X-rays of iodine) incident on the pixels 150 within a period of time. The pixels 150 may be configured to operate in parallel. For example, when one pixel 150 measures an incident X-ray photon, another pixel 150 may be waiting for an X-ray photon to arrive. The pixels 150 may not have to be individually addressable. Each of the X-ray detectors 102 may be configured to count the numbers of X-ray photons within the same period of time.

Each pixel 150 may be able to measure its dark current, such as before or concurrently with receiving each X-ray photon. Each pixel 150 may be configured to deduct the contribution of the dark current from the energy of the X-ray photon incident thereon.

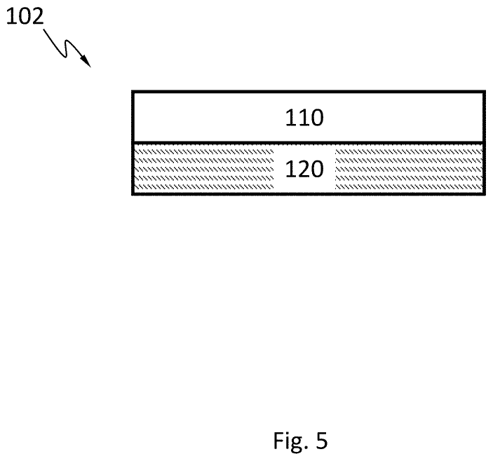

FIG. 5 schematically shows a cross-sectional view of the X-ray detector 102, according to an embodiment. The X-ray detector 102 may include an X-ray absorption layer 110 configured to generate an electrical signals responsive to photons of the characteristic X-rays incident thereon. In an embodiment, the X-ray detector 102 does not comprise a scintillator. The X-ray absorption layer 110 may include a semiconductor material such as, silicon, germanium, GaAs, CdTe, CdZnTe, or a combination thereof.

The X-ray detector 102 may include an electronics layer 120 for processing or analyzing the electrical signals incident X-ray photons generate in the X-ray absorption layer 110. The electronics layer 120 may be integrated with the absorption layer 110 into the same chip. Alternatively, the electronics layer 120 may be constructed on a separate semiconductor wafer different from the absorption layer 110 and bonded to the absorption layer 110. Examples of the X-ray absorption layer 110 and the electronics layer 120 may be found in a PCT Application PCT/CN2015/075950, the disclosure of which is incorporated by reference in its entirety.

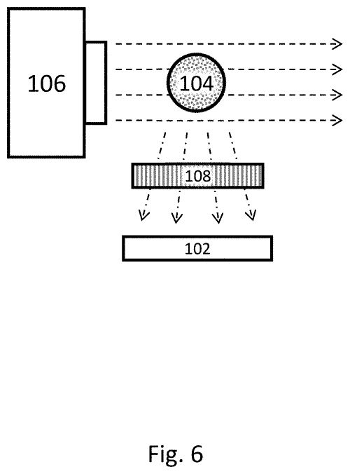

FIG. 6 schematically shows that the system 200 may include a collimator 108, according to an embodiment. The collimator 108 may be positioned between the object 104 and the detectors 102. The collimator 108 is configured to limit fields of view of the pixels 150 of the detectors 102. For example, collimator 108 may allow only X-rays with certain angles of incidence to reach the pixels 150. The range of angles of incidence may be <=0.04 sr, or <=0.01 sr.

The collimator 108 may be affixed on the detectors 102 or separated from the detectors 102. There may be spacing between the collimator 108 and the detectors 102. The collimator 108 may be movable or stationary relative to the detectors 102. The system 200 may include more than one collimator 108.

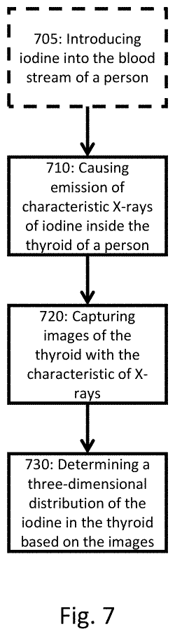

FIG. 7 shows a flowchart for a method, according to an embodiment. In optional procedure 705, iodine is introduced into the blood stream of the person. The iodine may be not radioactive. In procedure 710, emission of the characteristic X-rays of iodine inside the thyroid of a person is caused. For example, the emission of the characteristic X-rays may be a result of irradiating the thyroid with radiation that has sufficiently high energy. The radiation may be X-ray or gamma ray. In procedure 720, images of the thyroid are captured with the characteristic X-rays, using the X-ray detectors 102 positioned at different locations relative to the thyroid. In procedure 730, a three-dimensional distribution of the iodine in the thyroid is determined based on the images.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A system, comprising:
a plurality of X-ray detectors;
wherein the X-ray detectors are configured to be positioned at different locations relative to the thyroid of a person, and to capture images of the thyroid with characteristic X-rays of iodine.

2. The system of claim 1, further comprising a radiation source configured to irradiate the thyroid with radiation that causes iodine inside the thyroid to emit the characteristic X-rays.

3. The system of claim 1, wherein each of the X-ray detectors comprises an array of pixels, and is configured to count numbers of photons of the characteristic X-rays incident on the pixels within a period of time.

4. The system of claim 3, wherein each of the X-ray detectors is configured to count the numbers of X-ray photons within a same period of time.

5. The system of claim 3, wherein the pixels are configured to operate in parallel.

6. The system of claim 3, wherein each of the pixels is configured to measure its dark current.

7. The system of claim 3, wherein at least one of the X-ray detectors further comprises a collimator configured to limit fields of view of the pixels.

8. The system of claim 2, wherein energies of particles of the radiation are in the range of 30-40 keV.

9. The system of claim 2, wherein the radiation is X-ray or gamma ray.

10. The system of claim 1, wherein at least one of the X-ray detectors comprises an X-ray absorption layer configured to generate an electrical signal responsive to photons of the characteristic X-rays incident thereon.

11. The system of claim 10, wherein the X-ray absorption layer comprises silicon, germanium, GaAs, CdTe, CdZnTe, or a combination thereof.

12. The system of claim 1, wherein the X-ray detectors do not comprise a scintillator.

13. The system of claim 1, further comprising a processor configured to determine a three-dimensional distribution of the iodine in the thyroid, based on the images.

14. The system of claim 1, wherein the iodine is not radioactive.

15. A method, comprising:
causing emission of characteristic X-rays of iodine inside the thyroid of a person;
capturing images of the thyroid with the characteristic X-rays, using a plurality of X-ray detectors positioned at different locations relative to the thyroid;
determining a three-dimensional distribution of the iodine in the thyroid based on the images.

16. The method of claim 15, wherein causing emission of the characteristic X-rays comprises irradiating the thyroid with radiation that causes the emission of the characteristic X-rays.

17. The method of claim 16, wherein the radiation is X-ray or gamma ray.

18. The method of claim 15, wherein the iodine is not radioactive.

19. The method of claim 15, further comprising introducing the iodine into the blood stream of the person.

20. The method of claim 15, wherein each of the X-ray detectors comprises an array of pixels, and is configured to count numbers of photons of the characteristic X-rays incident on the pixels within a period of time.

21. The method of claim 20, wherein each of the X-ray detectors is configured to count the numbers within a same period of time.

22. The method of claim 20, wherein the pixels are configured to operate in parallel.

23. The method of claim 20, wherein each of the pixels is configured to measure its dark current.

24. The method of claim 20, wherein at least one of the X-ray detectors further comprises a collimator configured to limit fields of view of the pixels.

25. The method of claim 15, wherein at least one of the X-ray detectors comprises an X-ray absorption layer configured to generate an electrical signal responsive to photons of the characteristic X-rays incident thereon.

26. The method of claim 25, wherein the X-ray absorption layer comprises silicon, germanium, GaAs, CdTe, CdZnTe, or a combination thereof.

27. The method of claim 15, wherein the X-ray detectors do not comprise a scintillator.

28. The method of claim 15, wherein capturing the images comprises counting numbers of photons of the characteristic X-rays within a period of time.

29. The method of claim 28, wherein capturing the images comprises counting numbers of photons of the characteristic X-rays within a same period of time.
